(19) United States
(12) Patent Application Publication
(10) Pub. No.: US 2024/0277334 A1
(43) Pub. Date: Aug. 22, 2024

(54) SELF-CINCHING ADJUSTABLE LOOP SUTURE CONSTRUCT

(71) Applicant: Christian N. Anderson, Nashville, TN (US)

(72) Inventor: Christian N. Anderson, Nashville, TN (US)

(21) Appl. No.: 18/582,603

(22) Filed: Feb. 20, 2024

Related U.S. Application Data
(60) Provisional application No. 63/446,836, filed on Feb. 18, 2023.

(51) Int. Cl. A61B 17/06; A61B 17/04; A61F 2/08

(52) U.S. Cl. CPC A61B 17/06166; A61F 2/0811; A61B 2017/0496; A61F 2002/0817; A61F 2002/0852

(57) ABSTRACT

A self-cinching adjustable loop suture construct includes a suture strand including a first end, a second end and an adjustable loop. An end loop is disposed on the first end, and a continuous loop is disposed on the suture strand in the adjustable loop, wherein the continuous loop is moveable along the suture strand in the adjustable loop. A self-cinching section is disposed in the suture strand between the adjustable loop and the end loop. An anchor is disposed on the end loop. A shuttling suture is disposed on the continuous loop, and a passing suture is disposed on the end loop. The suture construct provides a self-cinching adjustable loop tension device for securing tissue during ligament repair or reconstruction that can be secured without the use of knots to maintain tension.

### CROSS-REFERENCES TO RELATED APPLICATIONS

This application claims benefit of and priority to U.S. Provisional Application No. 63/446,836 filed Feb. 18, 2023 entitled Self-Cinching Suture Devices and Methods, all of which is hereby incorporated by reference in its entirety.

### TECHNICAL FIELD

The present disclosure relates to devices and methods for orthopedic surgery, and more particularly to suture constructs for securing ligaments and tissue.

### BACKGROUND

Conventional suture constructs and associated methods for fixation of flexible suture material are difficult or ineffective for use with certain types of orthopedic procedures. Conventional suture constructs for ligament fixation often rely on one or more strands of flexible suture material secured to tissue, bone, anchors or other suture constructs to provide tension to one or more ligaments for repair or reconstruction. However, conventional suture constructs commonly rely on knots to secure the tag ends of suture material together or to secure the suture material to a structure. Such knots may loosen over time due to fatigue or cyclical loading of a joint. Additionally, conventional knots in suture material provide failure points and may be uncomfortable for a patient during the healing process.

Additionally, rigid conventional suture anchors commonly used with suture constructs for ligament fixation may be difficult to pass through tissue or bone tunnels due to the physical profile and dimensions of the anchor. Such limitations on rigid anchors constrain the sizes and shapes available for use in many orthopedic procedures, especially procedures which utilize a trans-osseous tunnel drilled through a bone, as the rigid anchor must be able to pass through the tunnel in some applications. Rigid fixation anchors may also lead to discomfort following a surgical procedure.

What is needed then, are improvements in suture constructs for repairing and reconstructing tissue in orthopedic surgical procedures.

### BRIEF SUMMARY

The present disclosure provides improved self-cinching adjustable loop suture constructs and associated methods for performing orthopedic surgery.

In some embodiments, the disclosure provides a suture construct apparatus, including a suture strand including a first end, a second end and an adjustable loop. An end loop is disposed on the first end, and a continuous loop disposed on the suture strand in the adjustable loop, wherein the continuous loop is moveable along the suture strand in the adjustable loop. A self-cinching section is disposed in the suture strand between the adjustable loop and the end loop. An anchor is disposed on the end loop in some embodiments.

In some embodiments, a shuttling suture is disposed on the continuous loop, and a passing suture is disposed on the end loop. The suture construct may be pulled through tissue, a bone tunnel, an anchor, another suture construct or other structure using the passing suture and/or the shuttling suture until the anchor is at a desired position for fixation.

One object of the present disclosure is to provide a suture construct with an anchor configured to maintain a compact profile while the suture construct is being pulled through tissue, a bone tunnel, anchor, another suture construct or other structure, and to self-deploy upon exiting the structure at a desired location.

Another objective of the present disclosure is to provide a self-cinching adjustable loop suture construct that may be tightened and maintain tension without the use of knots.

Yet another objective of the present disclosure is to provide a self-cinching adjustable loop suture construct with a compressible anchor that may retain a low profile during passage of the suture construct and expand to a larger profile upon tensioning of the suture construct after passage.

Another objective of the present disclosure is to provide a self-cinching adjustable loop suture construct for use in repair of the anterior cruciate ligament (ACL).

A further objective of the present disclosure is to provide a self-cinching adjustable loop suture construct for use during reconstruction of the anterior cruciate ligament (ACL).

Yet another objective of the present disclosure is to provide a self-cinching adjustable loop suture construct for use during a meniscal root repair in the knee.

A further objective of the present disclosure is to provide a self-cinching adjustable loop suture construct that can be pulled through tissue in a first direction, but self-deploys when tension is applied to the suture construct in the opposite direction.

Numerous other objects, advantages and features of the present disclosure will be readily apparent to those of skill in the art upon a review of the drawings and description as set forth below.

### DESCRIPTION OF THE DRAWINGS

FIG. 1 illustrates an embodiment of a self-cinching adjustable loop suture construct.

FIG. 2 illustrates a detail view of the embodiment of a self-cinching adjustable loop suture construct of FIG. 1.

FIG. 3 illustrates an alternative embodiment of a self-cinching adjustable loop suture construct with a passing suture and a shuttling suture.

FIG. 4 illustrates a detail view of the embodiment of a self-cinching adjustable loop suture construct of FIG. 3.

FIG. 5 illustrates a detail view of an embodiment of a self-cinching adjustable loop suture construct including an anchor disposed on the end loop.

FIG. 6 illustrates an alternative view of an embodiment of a self-cinching adjustable loop suture construct including an anchor disposed on the end loop.

FIG. 7 illustrates an embodiment of a self-cinching section of a self-cinching adjustable loop suture construct.

FIG. 8 illustrates an alternative embodiment of a self-cinching section of a self-cinching adjustable loop suture construct.

FIG. 9 illustrates an alternative embodiment of a self-cinching section of a self-cinching adjustable loop suture construct.

FIG. 10 illustrates an alternative embodiment of a self-cinching section of a self-cinching adjustable loop suture construct.

FIG. 11 illustrates an alternative embodiment of a self-cinching section of a self-cinching adjustable loop suture construct.

FIG. 12 illustrates an alternative embodiment of a self-cinching section of a self-cinching adjustable loop suture construct.

FIG. 13 illustrates an alternative embodiment of a self-cinching section of a self-cinching adjustable loop suture construct.

### DETAILED DESCRIPTION

The present disclosure provides various embodiments of improved self-cinching adjustable loop suture constructs for use during orthopedic surgery.

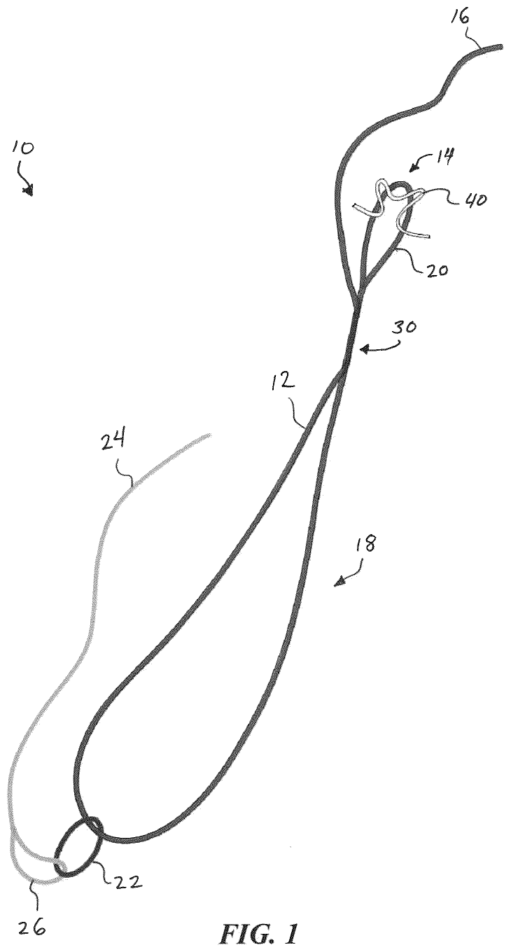

Referring to FIG. 1, an embodiment of a self-cinching adjustable loop suture construct 10 includes a suture strand 12 made of any suitable orthopedic flexible suture material, such as but not limited to a hollow-core suture, braided suture, flat suture tape, or other suitable suture material known in the art. Suture strand 12 includes a first end 14 and a second end 16. An end loop 20 is formed on the first end 14, and the second end 16 includes a free tag end that may be pulled for tensioning the construct. Second end 16 initially extends away from end loop 20 by a distance, makes a 180 degree turn back toward the end loop 20 forming an adjustable loop 18, and second end 16 is passed through its own suture strand 12 forming a self-cinching section 30. Second end 16 then exits self-cinching section 30 in a direction away from adjustable loop 18 terminating at a tag end.

The self-cinching section 30 is formed in some embodiments by passing second end 16 into suture strand 12, axially through the hollow core of suture strand 12 over a short length, and exiting suture strand 12 between self-cinching section 30 and end loop 20. As such, second end 16 may be pulled to tighten adjustable loop 18. During tightening, the suture material may slide through self-cinching section 30, thereby shortening adjustable loop 18 and tensioning the suture construct. Self-cinching section 30 provides a locking mechanism that maintains tension on the suture construct 10 when tightened.

Referring further to FIG. 1, in some embodiments, a free continuous loop 22 is disposed on the adjustable loop 18. Free continuous loop 22 forms a ring of suture material. Continuous loop 22 may include any suitable suture material such as hollow core suture, braided suture, flat suture tape, or any other suture material that can be formed in a continuous ring. In some embodiments, continuous loop 22 is formed by splicing suture material into a continuous ring. Suture strand 12 can slide freely through continuous loop 22 as adjustable loop 18 is tightened, and continuous loop may move along suture strand 12 inside adjustable loop 18.

A shuttling suture 24 is disposed on continuous loop 22. Shuttling suture 24 may be configured to pass the suture construct through an anchor, tissue, ligament, bone, another suture construct or other structure associated with performing an orthopedic procedure. Shuttling suture 24 in some embodiments is a sacrificial suture and is intended to be cut and removed from the suture construct 10 after implantation of suture construct 10. Shuttling suture 24 includes a shuttling suture loop 26 in some embodiments. Shuttling suture loop 26 passes through continuous loop 22 thereby securing shuttling suture 24 to continuous loop 22. Shuttling suture 24 includes any suitable flexible suture material, such but not limited to as a hollow-core suture, braided suture, flat suture tape, or other suture material. Shuttling suture loop 26 may be formed by splicing shuttling suture 24 back into itself in some embodiments.

In some embodiments, an anchor 40 is disposed on end loop 20 on first end 14 of suture strand 12. Anchor 40 includes any suitable anchor for securing suture construct 10 to a structure in tension, such as but not limited to suture tape, suture material, plastic, PEEK, a button, a screw, an implant, a plate, a graft or an orthopedic device such as but not limited to a needle, a bioresorbable anchor, implant, button or screw. In some embodiments, as shown in FIG. 1, anchor 40 includes a soft anchor formed of flexible suture tape material. As such, anchor 40 is a compressible material and may deform in shape while the suture construct is being pulled through tissue, a bone tunnel or other structure. Due to the compressible and resilient material of anchor 40 in some embodiments, anchor 40 may self-deploy to a larger profile and dimension upon exiting the structure at a desired fixation location. Anchor 40 comprising a soft, flexible material may also conform to the surface of the bone or structure against which it is applied better than a rigid suture button. For example, the bone surface adjacent a bone tunnel may be irregular, curved, rough and generally non-planar. By providing anchor 40 in the form of a soft, deformable anchor material, the anchor once deployed and tensioned engages the irregular bone surface in a compact profile, and the anchor 40 may conform in shape to the irregular surface geometry and texture of the bone, thereby enhancing fixation.

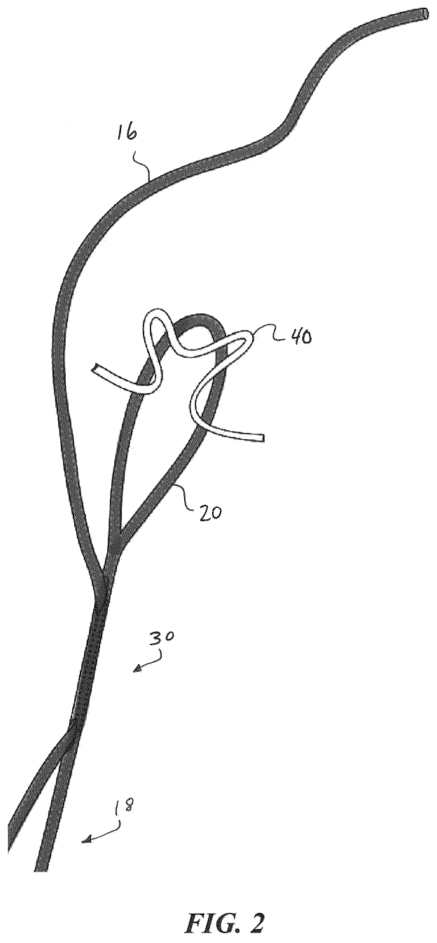

Referring to FIG. 2, in some embodiments, anchor 40 is formed by passing the suture material of end loop 20 transversely through the suture tape material of anchor 40 in multiple passes, thereby securing anchor 40 to the suture strand forming end loop 20. End loop 20 may be formed by splicing the suture material of the suture strand into itself, thereby forming a fixed loop after passing the suture strand transversely through the suture tape material of anchor 40.

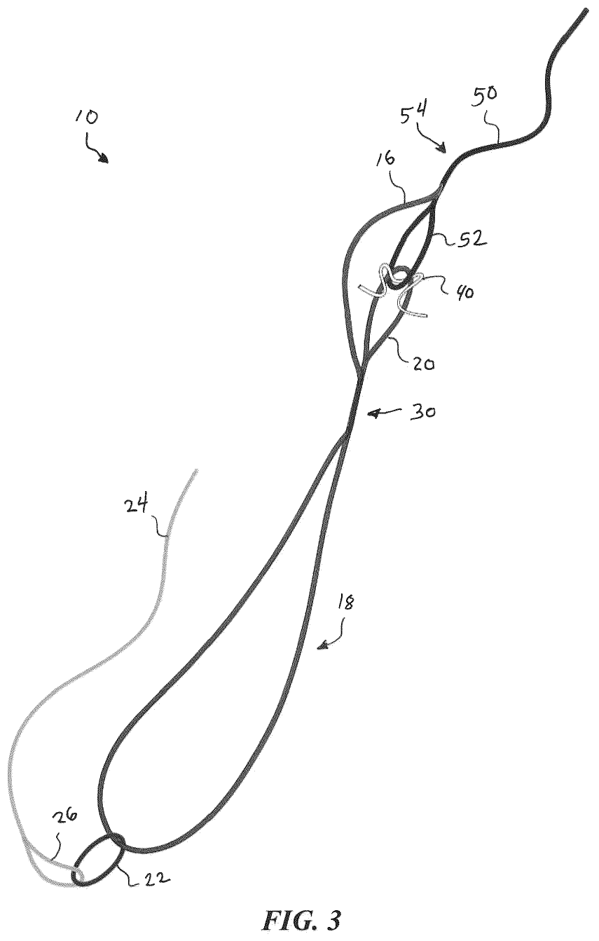

Referring to FIG. 3, an alternative embodiment of a suture construct 10 includes a the structure described previously with respect to FIG. 1, and also includes a passing suture 50 disposed on end loop 20 in addition to anchor 40. Passing suture 50 includes any suitable flexible suture material, such as but not limited to a hollow-core suture, braided suture, flat suture tape, or other suture material. Passing suture 50 may be configured to pass the suture construct through an anchor, tissue, ligament, bone, another suture construct or other structure associated with performing an orthopedic procedure. Passing suture 50 in some embodiments is a sacrificial suture and is intended to be cut and removed from the suture construct 10 after implantation of suture construct 10. Passing suture 50 includes a passing suture loop 52 in some embodiments. Passing suture loop 52 passes through end loop 20 thereby securing passing suture 50 to end loop 20. Passing suture loop 52 may be formed by splicing the suture material of passing suture 50 into itself.

The free tag end of second end 16 may be removably inserted into a passing suture sleeve 54 defined in passing suture 50 to temporarily conceal the tag end of second end 16 while passing the suture construct through tissue, bone, a ligament, anchor or other structure during a procedure. By inserting the tag end of second end 16 into the passing suture sleeve 54, the suture construct 10 may be more easily pulled without the tag end catching or becoming inadvertently displaced or damaged. Once the passing suture 50 is pulled to a desired position, the tag end of second end 16 may be removed from passing suture sleeve 54 by pulling the tag end free. In some embodiments, the tag end of second end 16 is inserted axially into the hollow core of the suture material of passing suture 50 which forms passing suture sleeve 54.

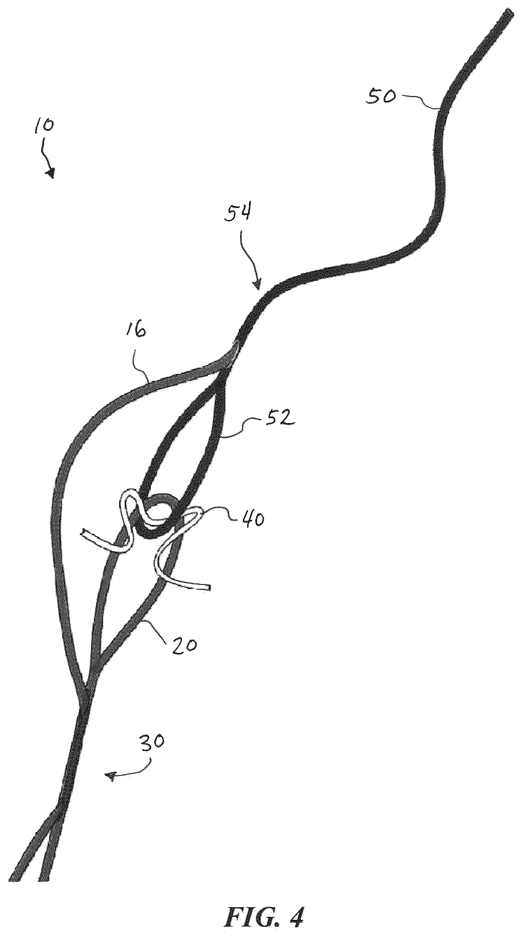

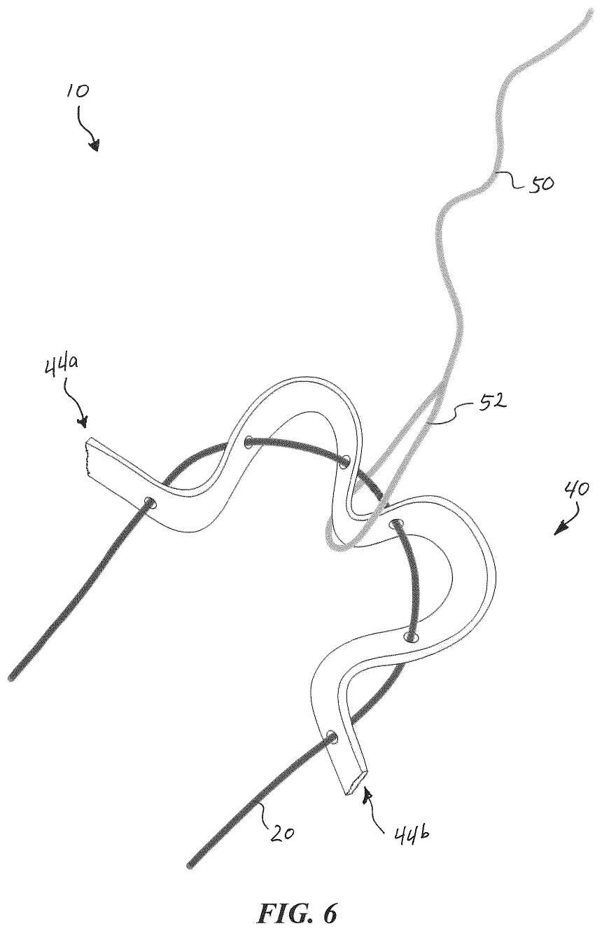

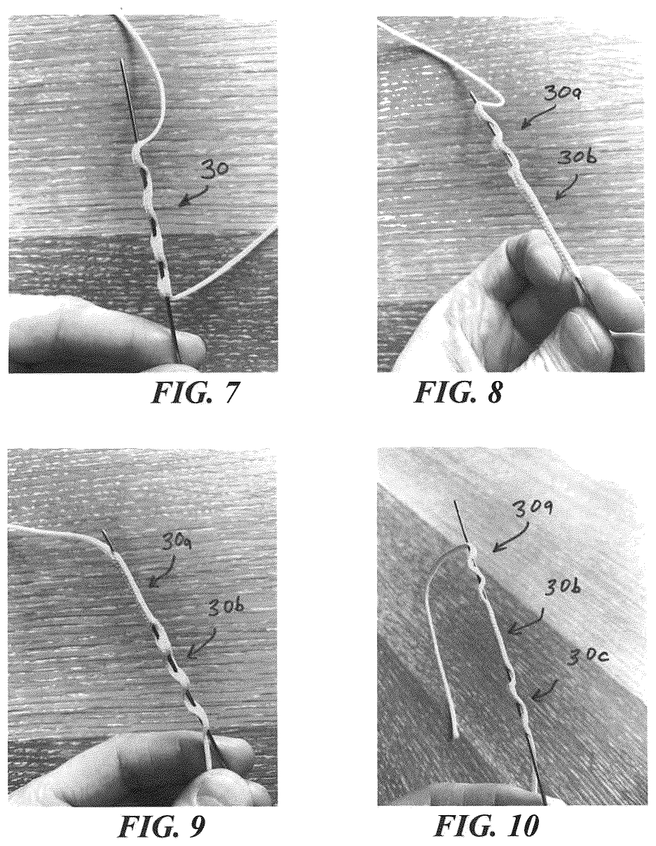

Referring to FIG. 4 and FIG. 6, in some embodiments, the passing suture loop 52 of passing suture 50 extends around both end loop 20 and anchor 40. As such, a portion of anchor 40 is disposed between the suture material of passing suture loop 52 and end loop 20.

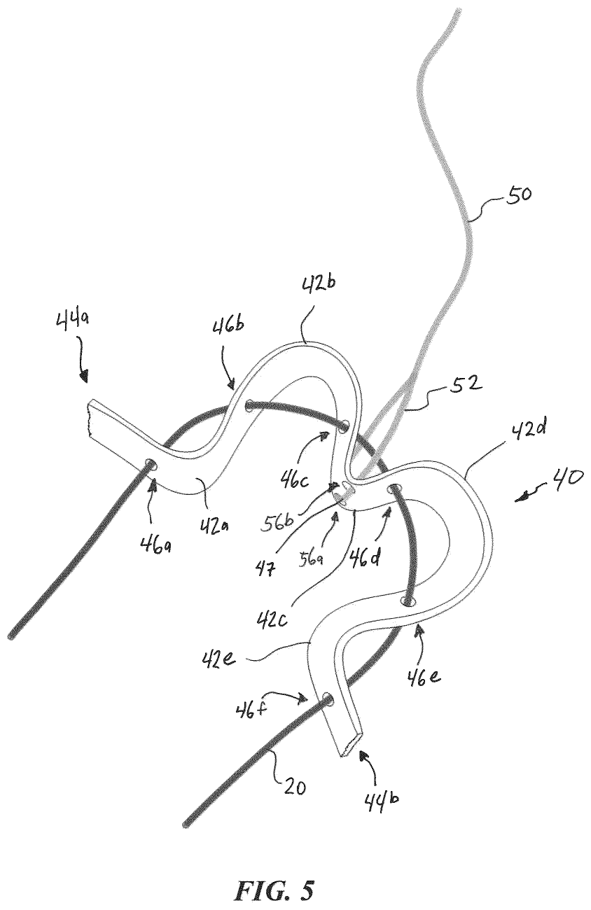

Referring to FIG. 5, in some embodiments, anchor 40 includes a substantially flat, flexible material disposed on end loop 20. Anchor 40 may include flexible, flat suture tape material in some embodiments. The suture strand forming end loop 20 is passed transversely through the body of anchor 40 a number of times, thereby securing anchor 40 to end loop 20. As shown in FIG. 5, anchor 40 includes a first pass 46a, a second pass 46b, a third pass 46c, a fourth pass 46d, a fifth pass 46e and a sixth pass 46f. Each pass includes a hole in the anchor where the suture strand of end loop 20 passes transversely through the material of anchor 40.

Multiple anchor loops are formed on anchor 40 when the suture strand of end loop 20 is passed through anchor 40. A first anchor loop 42a is formed on anchor 40 between first and second passes 46a, 46b. In some embodiments, first anchor loop 42a is oriented radially inwardly toward end loop 20 such that a first anchor end 44a projects radially away from end loop 20. As such, first anchor end 44a is configured to enhance engagement of anchor 40 with structure when the suture construct is deployed and tensioned.

A second anchor loop 42b is formed on anchor 40 between second pass 46b and third pass 46c. In some embodiments, second anchor loop 42b is oriented radially outwardly away from end loop 20. A third anchor loop 42c is formed on anchor 40 between third pass 46c and fourth pass 46d. In some embodiments, third anchor loop 42c is oriented radially inwardly toward end loop 20. A fourth anchor loop 42d is formed on anchor 40 between fourth pass 46d and fifth pass 46e. In some embodiments, fourth anchor loop 42d is oriented radially outwardly away from end loop 20. A fifth anchor loop 42e is formed on anchor 40 between fifth pass 46e and sixth pass 46f. In some embodiments, fifth anchor loop 42e is oriented radially inwardly toward end loop 20 such that a second anchor end 44b projects radially away from end loop 20 opposite first anchor end 44a. As such, second anchor end 44b is configured to enhance engagement of anchor 40 with structure when the suture construct is tensioned.

Referring further to FIG. 5, in some embodiments, passing suture 50 includes a passing suture loop 52 which also passes through anchor 40. The strand of material forming passing suture loop 52 passes around end loop 20, through a first hole 56a formed in anchor 40 between third pass 46c and fourth pass 46d, travels across the surface of anchor 40 forming a bridge 47 on the side of anchor 40 facing end loop 20, and exits through a second hole 56b in anchor 40 back toward passing suture 50. The connection of passing suture 50 to both anchor 40 and end loop 20 prevents anchor 40 from dislocating to an undesired position when the suture construct is being pulled through tissue, a ligament or a bone tunnel.

When suture construct 10 is being pulled through structure (such as tissue or a bone tunnel) via passing suture 50, first and second anchor ends 44a, 44b of anchor 40 may extend loosely along the suture strand in a linear direction toward adjustable loop 18 in a low profile. However, once the suture construct is cleared to the other side of the structure through which it is being pulled via passing suture 50 (such as through a bone tunnel), and tension is applied to the suture construct by pulling second end 16, anchor 40 may be deployed. In some applications, upon clearing a bone tunnel, first and second anchor ends 44a, 44b resiliently self-deploy and project radially outwardly away from the end loop 20 along the surface of anatomy such as a femoral cortex during ACL repair or reconstruction. As tension is applied to the suture construct, anchor 40 grips the femoral cortex and compresses in a direction away from adjustable loop 18 as first and second anchor ends 44a, 44b converge toward each other along end loop 20, thereby forming an occlusion against the adjacent tissue or bone which prevents the anchor from dislodging back through the tunnel. During use, when suture construct 10 is tensioned, the multiple anchor loops on anchor 40 fold toward third anchor loop 42c, which is generally along the midline of the anchor 40, thereby expanding the profile of the anchor 40 and providing a fixed point against which tension may be further applied to the suture construct 10.

Various embodiments of self-cinching section 30 are disclosed for providing the self-cinching features described herein. In a first embodiment, as shown in FIG. 2 and FIG. 4, a single pass axial self-cinching sleeve is formed by passing a suture strand axially through its hollow core suture material to form a locking mechanism. Referring to 7, in another embodiment, a suture material can be spliced in an entirely woven configuration wherein a suture strand is passed transversely through its suture material using a needle to form self-cinching section 30. Multiple transverse passes may be provided to achieve a desired locking effect, with more transverse passes generally creating a greater locking effect when the suture construct is tensioned. This configuration can be used with hollow core suture material or tape suture material. Referring to FIG. 8, in another embodiment, a suture material can be spliced in a single pass-woven configuration, wherein a woven portion includes suture material passing transversely through the body of the suture material in a first self-cinching section 30a and a single pass includes passing suture material axially through the hollow core of its suture material adjacent the woven portion forming a second self-cinching section 30b, which together form self-cinching section 30 along an axial length of the suture strand.

Referring to FIG. 9, in another embodiment, a suture material can be spliced in a woven-single pass configuration, which is a reverse of the configuration shown in FIG. 8, with first self-cinching section 30a being an axial single pass through the hollow core of the suture material forming a sleeve, and second self-cinching section 30b including multiple transverse passes of the suture strand through itself.

Referring to FIG. 10, an embodiment of a self-cinching section includes a woven-single pass-woven configuration including first and second woven sections and a single pass axial sleeve disposed between the woven sections, with first self-cinching section 30a including a woven section with multiple transverse passes, second self-cinching section 30b including an axial single pass through the hollow core of the suture material forming a sleeve, and third self-cinching section 30c including a woven section with multiple transverse passes.

Referring to FIG. 11, an embodiment of a self-cinching section includes a single pass-woven-single pass configuration including first and second axial sleeves and a woven section between the first and second sleeves. In this embodiments, first self-cinching section 30a includes an axial single pass through the hollow core of the suture material, second self-cinching section 30b includes a woven section with multiple transverse passes of the suture material through itself, and third self-cinching section includes an axial single-pass through the hollow core of the suture material.

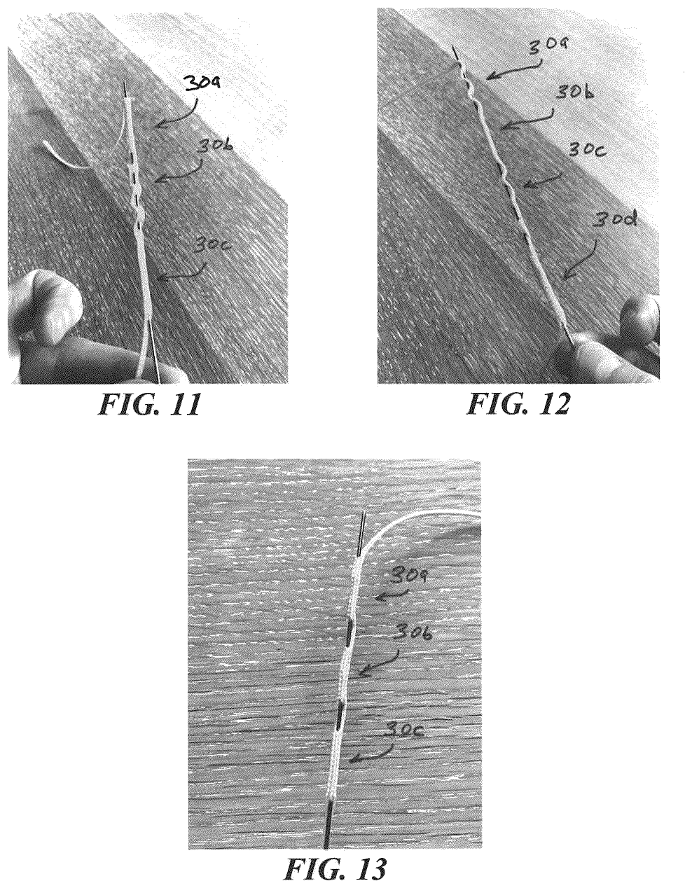

Referring to FIG. 12, an embodiment of a self-cinching section includes a single pass-woven-single pass-woven configuration. In this embodiment, first self-cinching section 30a and third self-cinching section 30c each include a woven section with multiple transverse passes of the suture material through itself, and second and fourth self-cinching sections 30b, 30d each include an axial single pass through the hollow core of the suture material.

Referring to FIG. 13, an embodiment of a self-cinching section includes multiple axial single pass sections of suture material passing through the hollow core of itself in first, second and third self-cinching sections 30a, 30b, 30c. Numerous other combinations of single pass, woven or single pass and woven can be combined to form a self-cinching section on suture construct.

Thus, although there have been described particular embodiments of the present invention of a new and useful SELF-CINCHING ADJUSTABLE LOOP SUTURE CONSTRUCT AND METHODS, it is not intended that such references to particular embodiments be construed as limitations upon the scope of this invention.

What is claimed is:

1. A suture construct apparatus, comprising:
a suture strand including a first end, a second end and an adjustable loop;
an end loop disposed on the first end;
a continuous loop positioned on the suture strand in the adjustable loop, wherein the continuous loop is moveable along the suture strand in the adjustable loop;
a self-cinching section disposed in the suture strand between the adjustable loop and the end loop;
an anchor disposed on the end loop;
a shuttling suture disposed on the continuous loop; and
a passing suture disposed on the end loop.

2. The apparatus of claim 1, further comprising:
a passing suture loop disposed on the passing suture, wherein the passing suture loop extends through the end loop.

3. The apparatus of claim 2, wherein the passing suture loop engages the anchor inside the end loop.

4. The apparatus of claim 3, further comprising:
a shuttling suture loop disposed on the shuttling suture.

5. The apparatus of claim 4, wherein the shuttling suture loop is disposed around the continuous loop.

6. The apparatus of claim 5, wherein the anchor is substantially flat.

7. The apparatus of claim 6, wherein the anchor is flexible.

8. The apparatus of claim 7, wherein the anchor comprises flexible flat suture tape.

9. The apparatus of claim 8, wherein the anchor includes first and second anchor ends extending radially away from the end loop.

10. The apparatus of claim 9, wherein the anchor comprises first, second, third, fourth and fifth anchor loops each extending away from the end loop.

11. The apparatus of claim 10, wherein the end loop passes transversely through the anchor between the first, second, third, fourth and fifth loops.

12. The apparatus of claim 11, wherein the passing suture extends around the anchor inside the end loop.

13. The apparatus of claim 11, wherein the passing suture extends transversely through the anchor inside the end loop.

14. The apparatus of claim 5, wherein the self-cinching section comprises the suture strand extending axially through itself.

15. The apparatus of claim 5, wherein the self-cinching section comprises the suture strand extending transversely through itself.

16. The apparatus of claim 5, wherein the self-cinching section comprises the suture strand extending axially and transversely through itself.
